(19) United States
(12) Patent Application Publication
(10) Pub. No.: US 2026/0354801 A1
(43) Pub. Date: Nov. 12, 2026

(54) CONTRAST-ENHANCED ULTRASOUND IMAGE QUALITY CONTROL

(71) Applicant: KONINKLIJKE PHILIPS N.V., EINDHOVEN (NL)

(72) Inventors: Jingping Xu, Shanghai (CN); Zhouye Chen, Shanghai (CN); Thanasis Loupas, Kirkland, WA (US); Qizhong Lin, Shanghai (CN); Nancy Wang, Shanghai (CN); Ling Tong, Shanghai (CL); Jinning Chen, Shanghai (CN)

(21) Appl. No.: 19/490,813

(22) PCT Filed: Jun. 5, 2024
(86) PCT No.: PCT/EP2024/065404

§ 371 Date: Dec. 5, 2025

(30) Foreign Application Priority Data
Jun. 9, 2023 (WO) PCT/CN2023/099345

(51) Int. Cl. A61B 8/00; A61B 8/08; G06T 5/73; G06T 7/00

(52) U.S. Cl. CPC A61B 8/5276; A61B 8/085; A61B 8/481; G06T 5/73; G06T 7/0012; G06T 2207/10132; G06T 2207/30096; G06T 2207/30168

(57) ABSTRACT

A system for classifying ultrasound images includes a processor and a tangible, non-transitory computer-readable medium that stores instructions. The instructions cause the system to apply motion compensation to a region covering a targeted suspected lesion in each ultrasound frame of interest among the CEUS images to ensure that the lesion remains at approximately the same image location for all ultrasound frames of interest among Contrast-Enhanced Ultrasound (CEUS) images, to generate a time-intensity curve (TIC) based on the motion-compensated ultrasound frames of interest among the CEUS images, to apply an organ-specific TIC model to the TIC to obtain a fitted TIC (FTIC), to compute a normalized fitting error (NFE) based on a difference between the TIC and the FTIC; compare the normalized fitting error to a first predetermined threshold for a first classification, and to obtain an out-of-plane frame ratio (ratio-TIC) for a second classification.

### BACKGROUND

Contrast-enhanced ultrasound (CEUS) is an ultrasound imaging technique used in a variety of clinical applications. Compared to conventional ultrasound, a much longer learning curve exists for beginners to obtain high quality CEUS examinations. Operators must gain adequate knowledge and hands-on-training with a sufficient number of examinations with various pathologies. One application for CEUS is for soft-tissue organs where tumor characterization is required. In the case of liver imaging, there are usually 10 sub-types of diseases. If 15 typical cases are needed to identify one sub-type well, then the operator should perform at least 150 CEUS examinations in total with guidance from experienced ultrasound doctors or radiologists during a learning phase. Hepatocellular carcinoma (HCC) diagnosis is one of the most important clinical applications. However, it could be extended to other clinical applications with a suitable modification in selecting an organ-specific “standardized” model.

CEUS can detect the nonlinear signals received from microbubbles which circulate in the blood stream after an intravenous injection of an ultrasound contrast agent. As such CEUS imaging allows for documentation of tissue perfusion due to comparatively slow flow at the capillary level, as well as visualizing relatively fast blood flow in arteries and veins. As a result, CEUS is capable at providing dynamic visualization of blood flow at both the macro- and micro-circulation levels. Among other clinical applications, CEUS imaging mode is recommended in the diagnosis and treatment monitoring of lesions on the liver, which may be malignant.

A sonographer typically operates a probe to gather images and loops that can span a few minutes. The representative images and loops gathered by the sonographer are often then sent to another location for review of the data by a radiologist or other trained clinician, for example. In some cases, a radiologist/clinician performs the sonogram, gathering/evaluating frames and loops used in diagnosis and treatment of medical condition. Moreover, radiologists/clinicians typically review cases in a remote workstation without being present during the CEUS exam which is the case at radiology departments in USA. So the data from the procedure must be stored and transmitted from the location of the sonographer to the radiologist/clinician. This data transfer can be challenging due to the comparatively long duration of the acquired CEUS sequences.

During a CEUS procedure, scans are often taken in a two dimensional plane through the portion of the body (e.g., the liver) being examined. A large number of frames and loops are gathered during the procedure and are sent for review by a trained clinician such as a radiologist/clinician. As will be appreciated, when a sonographer is taking a scan of a region of interest (ROI), there are many sources of movement that can impact the quality of the images being gathered. For example, movement of the patient due to breathing can result in a shift in the location of the image plane, resulting images out of the image plane of the current scan, and ultimately in images lesser quality and unproductive scans. CEUS examination involves multi-tasking where the operator (ultrasound doctor/sonographer) needs to take care of three major components: a) keep the ultrasound probe still and stable at the same position, i.e. targeted region of interest (ROI) for a few minutes; b) make sure that the patient under investigation is controlling his/her breathing well during the entire examination; c) watch the CEUS sequence for overall quality check to extract diagnostic information. Currently, a CEUS quality check is performed by visually observing the CEUS loops during and after the examination. The visual quality check, however, is highly observer-dependent, and is a time-consuming process. All three inter-connected tasks are complicated for inexperienced operator, thus bringing the limitations of current CEUS workflow: There is the risk especially for inexperienced operators that the acquired CEUS loop data is not of sufficient quality and, therefore, an additional contrast agent injection is required for correct diagnosis. There is a risk especially for inexperienced sonographer that the CEUS loop is found to be of insufficient quality when reviewed by a radiologist, thus the patient may need to return for a second examination.

Artifacts due to out-of-plane motion remain when using known advanced CEUS imaging systems. Of the comparatively large amount of image data gathered in a scan, much of the data can be out-of-plane and of undesirable quality due to motion during a CEUS scan. These data are often stored in memory, and are transmitted to the clinician for reviewing. As will be appreciated, more stored data or transmitted data, or both, places a burden on the computer system used to store, transmit and share the image data from the scan. These large amounts of out-of-plane image data, which are of lesser quality and thus not useful to the clinician reviewing the images, are stored in ever-scarce memory. Moreover, the clinician reviewing the scans from a CEUS procedure has to sort through many frames to find the part(s) where the lesion or organ of interest is in-plane to properly assess the patient's condition. As such, not only are out-of-plane image data a drain on memory resources, but also they occupy the clinician's time during review of the CEUS procedure.

What is needed is a system that overcomes at least the noted drawbacks of known systems set forth above.

### SUMMARY

According to an aspect of the present disclosure, a system for classifying ultrasound images includes a processor, and a tangible, non-transitory computer-readable medium. The tangible, non-transitory computer-readable medium stores instructions. When executed by the processor, the instructions cause the system to: apply motion compensation to a region covering a targeted suspected lesion in each ultrasound frame of interest among Contrast-Enhanced Ultrasound (CEUS) images to ensure that the lesion remains at approximately the same image location for all ultrasound frames of interest among the CEUS images; generate a time-intensity curve (TIC) based on the motion-compensated ultrasound frames of interest among the CEUS images; apply an organ-specific TIC model to the TIC to obtain a fitted TIC (FTIC); compute a normalized fitting error (NFE) based on a difference between the TIC and the FTIC; compare the normalized fitting error to a first predetermined threshold; when the normalized fitting error is larger than the first predetermined threshold, classify the CEUS images as low-quality; when the normalized fitting error is not larger than the first predetermined threshold, obtain an out-of-plane frame ratio (ratio-TIC); compare the out-of-plane frame ratio to a second predetermined threshold; when the out-of-plane frame ratio is larger than the second predetermined threshold, classify the CEUS images as low-quality; and when the out-of-plane frame ratio is not larger than the second predetermined threshold, classify the CEUS images as high-quality.

According to another aspect of the present disclosure, a tangible, non-transitory computer-readable medium stores instructions. When executed by a processor, the instructions cause the processor to: apply motion compensation to a region covering a targeted suspected lesion in each ultrasound frame of interest among Contrast-Enhanced Ultrasound (CEUS) images to ensure that the lesion remains at approximately the same image location for all ultrasound frames of interest among the CEUS images; generate a time-intensity curve (TIC) based on the motion-compensated ultrasound frames of interest among the CEUS images; apply an organ-specific TIC model to the TIC curve to obtain a fitted TIC (FTIC); compute a normalized fitting error (NFE) based on a difference between the TIC and the FTIC; compare the normalized fitting error to a first predetermined threshold; when the normalized fitting error is larger than the first predetermined threshold, classify the CEUS images as low-quality; when the normalized fitting error is not larger than the first predetermined threshold, obtain an out-of-plane frame ratio (ratio-TIC); compare the out-of-plane frame ratio to a second predetermined threshold; when the out-of-plane frame ratio is larger than the second predetermined threshold, classify the CEUS images as low-quality; and when the out-of-plane frame ratio is not larger than the second predetermined threshold, classify the CEUS images as high-quality.

According to another aspect of the present disclosure, a method of classifying ultrasound images includes: applying motion compensation to a region covering a targeted suspected lesion in each ultrasound frame of interest among Contrast-Enhanced Ultrasound (CEUS) images to ensure that the lesion remains at approximately the same image location for all ultrasound frames of interest among the CEUS images; generating a time-intensity curve (TIC) based on the motion-compensated ultrasound frames of interest among the CEUS images; applying an organ-specific TIC model to the TIC to obtain a fitted TIC (FTIC); computing a normalized fitting error (NFE) based on a difference between the TIC and the FTIC; comparing the normalized fitting error to a first predetermined threshold; when the normalized fitting error is larger than the first predetermined threshold, classifying the CEUS images as low-quality; when the normalized fitting error is not larger than the first predetermined threshold, obtaining an out-of-plane frame ratio (ratio-TIC); comparing the out-of-plane frame ratio to a second predetermined threshold; when the out-of-plane frame ratio is larger than the second predetermined threshold, classifying the CEUS images as low-quality; and when the out-of-plane frame ratio is not larger than the second predetermined threshold, classifying the CEUS images as high-quality.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of an imaging system for contrast-enhanced ultrasound image quality control, according to a representative embodiment.

FIG. 2A illustrates a method for contrast-enhanced ultrasound image quality control, in accordance with a representative embodiment.

FIG. 2B illustrates another method for contrast-enhanced ultrasound image quality control, in accordance with a representative embodiment.

FIG. 3 illustrates another method for contrast-enhanced ultrasound image quality control, in accordance with a representative embodiment.

FIG. 4 illustrates a visualization of an example of a number of detected out-of-frame planes relative to a number of frames in contrast-enhanced ultrasound image quality control, in accordance with a representative embodiment.

FIG. 5 illustrates another method for contrast-enhanced ultrasound image quality control, in accordance with a representative embodiment.

FIG. 6A illustrates a visualization of correlation coefficient relative to a number of frames in contrast-enhanced ultrasound image quality control, in accordance with a representative embodiment.

FIG. 6B illustrates a visualization of a time-intensity curve relative to a number of frames in contrast-enhanced ultrasound image quality control, in accordance with a representative embodiment.

FIG. 7A illustrates another visualization of correlation coefficient relative to a number of frames in contrast-enhanced ultrasound image quality control, in accordance with a representative embodiment.

FIG. 7B illustrates another visualization of a time-intensity curve relative to a number of frames in contrast-enhanced ultrasound image quality control, in accordance with a representative embodiment.

FIG. 8 illustrates a computer system, on which a method for contrast-enhanced ultrasound image quality control is implemented, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, a quality check module for contrast-enhanced ultrasound workflow improvement may improve the efficiency of clinical practices, particularly for the diagnosis of liver lesions. The quality check module may reduce the workload for ultrasound doctors/sonographers during CEUS examination and for radiologists during CEUS examination review and report. The quality check module may automatically check the quality of CEUS loops. The time and effort for a CEUS examination procedure for both operator (ultrasound doctor or sonographer) and patient may be reduced insofar as the quality check module helps guarantee the quality of each CEUS examination, as it can help ultrasound doctors/sonographers keep the ultrasound probe still and stable while ensuring the patient is controlling his/her breathing well during the entire examination; helps the ultrasound doctors/sonographers determine when another contrast agent injection is required before the patient leaving the exam room, which will potentially save a patient recall exam; and helps save the time of radiologists as only high quality CEUS loops are transferred to the picture archiving and communication system (PACS).

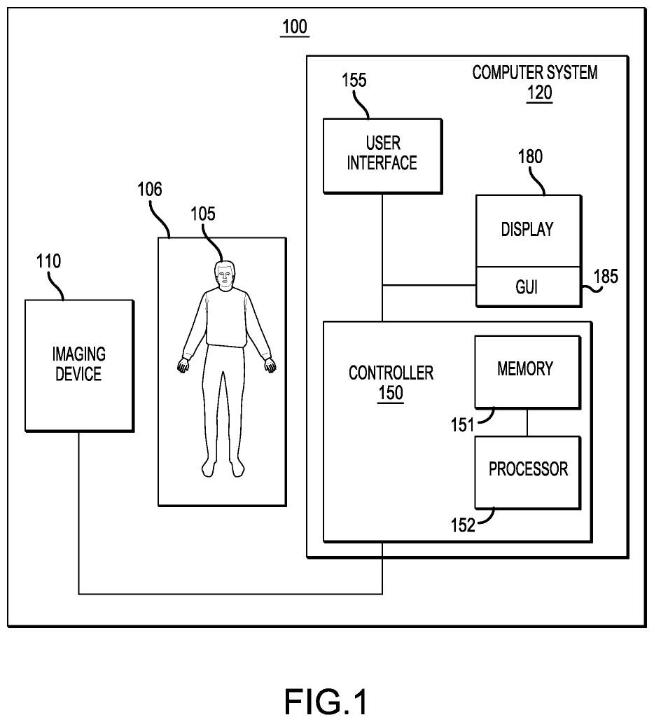

FIG. 1 is a simplified block diagram of an imaging system for contrast-enhanced ultrasound image quality control, according to a representative embodiment.

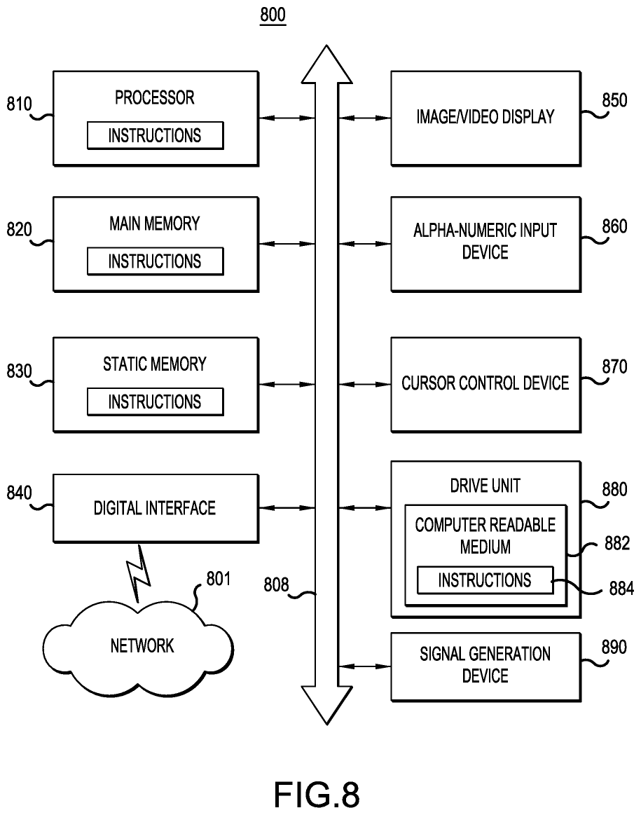

The imaging system 100 in FIG. 1 is a system for contrast-enhanced ultrasound image quality control and includes components that may be provided together or that may be distributed. The imaging system 100 includes an imaging device 110 and a computer system 120. The computer system 120 includes a controller 150, a user interface 155, and a display 180. The controller 150 includes a memory 151 that stores instructions and a processor 152 that executes the instructions. The display 180 includes a graphical user interface 185. The display 140 may also include a speaker such as a loudspeaker (not shown) to provide audible feedback. The computer system 120 may be provided as elements of an ultrasound base and an accompanying display (i.e., the display 180), or may be provided as elements of a dedicated computer system for contrast-enhanced ultrasound image quality control. The computer system 120 may process contrast-enhanced ultrasound images of a region of interest in a patient 105 on a table 106. The contrast-enhanced ultrasound images processed by the computer system 120 are captured by the imaging device 110. The imaging device 110 is illustratively an ultrasound imaging system capable of providing a contrast-enhanced ultrasound (CEUS) image scan of a region of interest (ROI) of the patient 105. The computer system 120 receives image data from the imaging device 110, and stores and processes the image data according to representative embodiments described herein. A computer system 800 that may be used to implement the computer system 120 is shown in and described with respect to FIG. 8, though the computer system 120 may include more or fewer elements than shown in FIG. 8.

The controller 150 may perform some of the operations described herein directly and may implement other operations described herein indirectly. For example, the controller 150 may indirectly control operations such as by generating and transmitting content to be displayed on the display 180. The controller 150 may directly control other operations such as logical operations performed by the processor 152 executing instructions from the memory 151 based on input received from electronic elements and/or users via the interfaces. Accordingly, the processes implemented by the controller 150 when the processor 152 executes instructions from the memory 151 may include steps not directly performed by the controller 150.

The memory 151 stores instructions executable by the processor 152. When executed, and as described more fully below, the instructions cause the controller 150 to allow the user to perform different steps using the graphical user interface 185 (GUI) or the user interface 155, or both, and, among other tasks, to initialize an ultrasound imaging device comprising a transducer. In addition, the controller 150 may implement additional operations based on executing instructions, such as instructing or otherwise communicating with another element of the computer system 120, including the display 180, to perform one or more of the processes described herein.

The memory 151 may include a main memory and/or a static memory, where such memories may communicate with each other and the other elements of the controller 150 via one or more buses. The memory 151 stores instructions used to implement some or all aspects of methods and processes described herein. As will become clearer as the present description continues, the instructions stored in memory 151 may be referred to as “modules,” with different modules comprising executable instructions, which when executed by the processor 152, carry out the various functions described in connection with various representative embodiments described below.

The memory 151 may be implemented by any number, type and combination of random access memory (RAM) and read-only memory (ROM), for example, and may store various types of information, such as software algorithms, which serves as instructions, which when executed by a processor cause the processor to perform various steps and methods according to the present teachings. Furthermore, updates to the methods and processes described herein may also be provided to the computer system 120 and stored in memory 151.

The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. The memory 151 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 151 may store software instructions and/or computer readable code that enable performance of various functions. The memory 151 may be secure and/or encrypted, or unsecure and/or unencrypted.

“Memory” is an example of computer-readable storage media, and should be interpreted as possibly being multiple memories or databases. The memory or database for instance may be multiple memories or databases local to the computer system 120, and/or distributed amongst multiple computer systems or computing devices, or disposed in the ‘cloud’ according to known components and methods. A computer readable storage medium is defined to be any medium that constitutes patentable subject matter under 35 U.S.C. § 101 and excludes any medium that does not constitute patentable subject matter under 35 U.S.C. § 101. Examples of such media include non-transitory media such as computer memory devices that store information in a format that is readable by a computer or data processing system. More specific examples of non-transitory media include computer disks and non-volatile memories.

The processor 152 is representative of one or more processing devices, and is configured to execute software instructions stored in memory 151 to perform functions as described in the various embodiments herein. The processor 152 may be implemented by field programmable gate arrays (FPGAs), application specific integrated circuits (ASICs), systems on a chip (SOC), a general purpose computer, a central processing unit, a computer processor, a microprocessor, a graphics processing unit (GPU), a microcontroller, a state machine, programmable logic device, or combinations thereof, using any combination of hardware, software, firmware, hard-wired logic circuits, or combinations thereof. Additionally, any processing unit or processor described herein may include multiple processors such as multicore processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems, such as in a cloud-based or other multi-site application. The term computing device should also be interpreted to include a collection or network of computing devices each including a processor or processors. Modules have software instructions to carry out the various functions using one or multiple processors that may be within the same computing device or which may be distributed across multiple computing devices.

The display 180 may be local to the controller 150 or may be remotely connected to the controller 150. The display 180 may be connected to the controller 150 via a local wired interface such as an Ethernet cable or via a local wireless interface such as a Wi-Fi connection. The display 180 may be interfaced with other user input devices by which users can input instructions, including mouses, keyboards, thumbwheels and so on. The display 180 may be a monitor such as a computer monitor, a television, a liquid crystal display (LCD), a light emitting diode (LED) display, a flat panel display, a solid-state display, or a cathode ray tube (CRT) display, or an electronic whiteboard, for example. The display 180 may also provide the graphical user interface 185 for displaying and receiving information to and from the user.

The user interface 155 may include a user and/or network interface for providing information and data output by the controller 150 to the user and/or for receiving information and data input by the user. That is, the user interface 155 enables the user to operate the imaging device 110 as described herein, and to schedule, control or manipulate aspects of the imaging system 100 of the present teachings. Notably, the user interface 155 enables the controller 150 to indicate the effects of the user's control or manipulation. The user interface 155 may include one or more of ports, disk drives, wireless antennas, or other types of receiver circuitry. The user interface 155 may further connect one or more interface devices, such as a mouse, a keyboard, a mouse, a trackball, a joystick, a microphone, a video camera, a touchpad, a touchscreen, voice or gesture recognition captured by a microphone or video camera, for example.

Notably, the controller 150, the display 180, the graphical user interface 185 and the user interface 155 may be located away from (e.g., in another location of a building, or another building) the imaging device 110 operated by a sonographer. The controller 150, the display 180, the graphical user interface 185 and the user interface 155 may be, for example, located where the radiologist/clinician is located. Notably, however, additional controllers, displays, GUI and user interfaces may be located near the sonographer and are useful in effecting the various functions of the imaging device 110 needed to complete the CEUS scans contemplated by the present teachings.

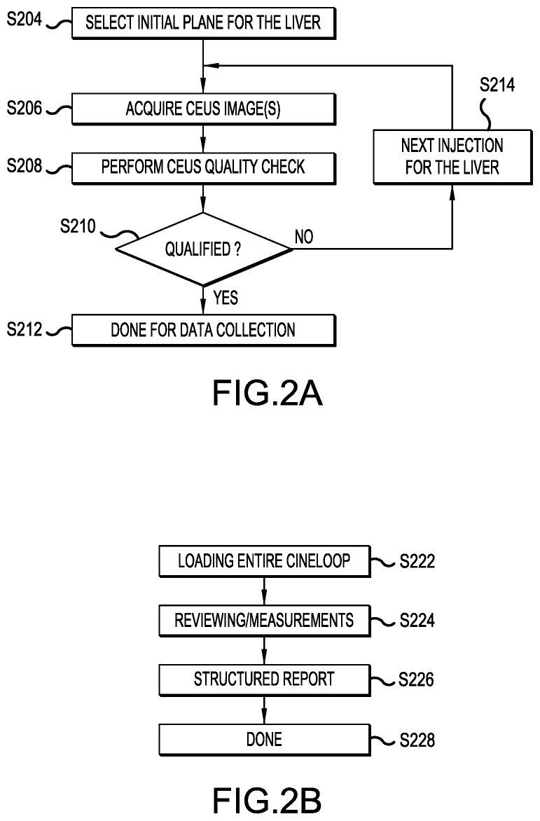

FIG. 2A illustrates a method for contrast-enhanced ultrasound image quality control, in accordance with a representative embodiment. FIG. 2B illustrates another method for contrast-enhanced ultrasound image quality control, in accordance with a representative embodiment.

FIG. 2A is a flow chart of a method of collecting images using the imaging system 100 of FIG. 1 to provide frames for a clinician for review according to a representative embodiment. Various aspects and details of the method are common to those described in connection with representative embodiments of FIG. 1. These common aspects and details may not be repeated to avoid obscuring the presently described representative embodiment.

Referring to FIG. 2A, an initial plane for imaging the liver is selected at S204 and a CEUS image is acquired at the initial plane at S206. The user interface 155 in FIG. 1 may be used to select a region of interest for a targeted lesion from the CEUS cine-loop at S204. That is, at S204 the sonographer begins a CEUS scan at an initial location, such as at a lesion on the liver. In performing the scan, the imaging device 110 captures one or more image(s) of a two-dimensional image plane (sometimes referred to as a slice), which is the initial plane, and the CEUS image(s) is/are acquired at the initial plane. The initial plane is located at a portion of the body selected for imaging, which for illustrative purposes may be a targeted lesion region at the middle of the ultrasound image. The CEUS image is acquired by putting the probe at a suitable position/orientation, then collecting incoming frames over the entire examination period of 3 to 6 minutes. As used herein and as described more fully below, an image taken at or close to the desired image plane from where the sonographer is attempting to gather image data is referred to as being an in-plane (IP) image and includes the full region for the lesion to be examined, and is desirable for further review to aid in diagnosis or treatment. Notably, these desired in-plane frames may be referred to herein as optimized frames at least because they provide the radiologist/clinician with frames most useful in diagnosing and treating a patient, and do not include out-of-plane (OOP) frames, which are not only less useful in diagnosis and treatment of a patient, but also, if provided to the radiologist/clinician, may cause the radiologist/clinician to be burdened with reviewing a comparatively large number of less than optimal frames from the CEUS procedure.

However, and as described more fully below, relative movement of the patient and imaging device can cause the imaging device 110 to capture an image in another image plane that is not the same as the desired initial plane. For example, when the sonographer is attempting to capture an image at the selected location (e.g., the targeted lesion region), movement of the patient (e.g., caused by breathing) or an unintended movement of the imaging device 110 by the sonographer, the imaging device 110 will have moved relative to the selected location. This will cause the imaging device 110 to capture an ultrasound image from another plane different from the initial plane. A desired in-plane image, based on certain factors discussed more fully below, taken at another plane that is too far from the initial plane is referred to herein as being an OOP image, and is not desirable. According to various aspects of the present teachings described in connection with representative embodiments below, OOP images that are deemed too far out of the initial plane may not be included in the images provided for review by a radiologist or similarly trained clinician. By one measure, in an OOP a significant portion (e.g., 70%-100%) of the targeted region for the lesion is lost in the current image frame.

After completion of S206, the method proceeds to S208 for performing a CEUS quality check. For example, the controller 150 may receive the ultrasound images acquired in S206 by the imaging device 110. The ultrasound images may be stored in the memory 151 and analyzed by the processor 152 executing a quality check module to check the quality of the ultrasound images gathered (e.g., in the 3-6 minute portion of the procedure as alluded to above). The quality check module implemented at S208 may target HCCs, but may also be adapted to target other types of liver lesions. The quality check module is configured to check CEUS images for quality, and may include two or more algorithms that execute distinct processing methods. The first algorithm may be a time-intensive curve (TIC) algorithm configured to perform TIC analysis and the second algorithm may be a cross-correlation coefficient algorithm configured to perform NCCC (normalized cross-correlation coefficient) analysis. The second algorithm may use an NCCC method to determine an out-of-plane frame ratio (ratio-NCCC) as described herein. The out-of-plane frame ratio (ratio-NCCC) may be computed between the number of out-of-plane frames and a total number of frames in the motion compensated loop of the CEUS images.

The first algorithm may use a TIC method to determine CEUS quality by obtaining normalized fitting error (NFE) or the out-of-plane frame ratio (ratio-TIC) described herein.

In some embodiments, the computer system 120 in FIG. 1 may include an additional user interface, such as on the graphical user interface 185. The additional user interface may be provided to help the sonographer or ultrasound doctor determine CEUS examination quality at S208 by combing two types of parameters from the first algorithm implementing the TIC method and the second algorithm implementing the NCCC method. A regression model may be used to comb the two types of parameters. In some embodiments, the controller 150 may take in both ultrasound data in DICOM format as well as raw data or radiofrequency (RF) signals, and the quality check performed at S208 may be implemented on-cart in an ultrasound cart which includes the controller 150 to improve CEUS workflow.

At S210, the sonographer determines if the image data acquired is sufficient for a complete review and analysis of the condition of the anatomy being imaged. When the sonographer determines that enough image data have been acquired (S210=Yes), the method proceeds to S212 where the collected data are stored, or transmitted to another location for storage and review, or both, and the data collection in FIG. 2A is complete.

When the sonographer determines that more image data is required (S210=No), the method continues at S214. Here a second contrast agent may be needed for the current plane or the next plane of the liver where the appearance of perfusion is not clear during the first injection period. The method then returns to S206, and the procedure is repeated until the sonographer determines at S210 that the image data acquired is sufficient for a complete review and analysis of the condition of the anatomy being imaged. The method then proceeds to S212 where the collected data are stored, or transmitted to another location for storage and review, or both. As described more fully below, OOP images that are not useful for the desired imaging procedure are removed and not stored at S212. Rather the image data that are stored at S212 comprise only images that are beneath one or more thresholds set for OOP images.

FIG. 2B is a flow chart of a method of reviewing all cinematic loops (cine loops) collected in the method of FIG. 2A by a radiologist or other trained clinician in accordance with a representative embodiment. Various aspects and details of the method are common to those described in connection with representative embodiments of FIGS. 1 and 2A. These common aspects and details may not be repeated to avoid obscuring the presently described representative embodiment.

At S222 the entire imaging procedure, including entire cineloops and notes from the sonographer are loaded for review by a radiologist/clinician. Notably, the CEUS data acquired during an exam may consist of multiple cineloops acquired at different times as opposed to a single cineloop covering the entire exam time. By way of illustration, the imaging procedure loaded at S222 may be initially stored on a suitable memory device and transported to another location wherein the sonographer is located. Alternatively, the entire imaging procedure gathered at S212 may be transmitted (e.g., by a wired or wireless communication link) and loaded at S222 for review by the radiologist/clinician. As will be appreciated, and as will become clearer as the present description continues, by the present teachings, only the in-plane images are stored and transmitted for loading at S222. Beneficially, compared to known systems that include OOP and in-plane image data for loading for review by the radiologist/clinician, only the in-plane images are stored or transmitted for loading at S222. This of course reduces the memory requirements of stored image data, or bandwidth requirements for transmitted image data, or both. As such, and among other benefits, the present teachings reduce the memory requirements, or the bandwidth requirements, or both, for the collection of image data to be reviewed by the radiologist/clinician.

At S224, the image data loaded at S222 are reviewed by the radiologist/clinician and measurement are taken by the radiologist/clinician from the in-plane images. Beneficially, because only the in-plane images are stored or transmitted for loading at S222, the radiologist/clinician does not have to review less than desirable images (OOP images) at S224. By contrast, image review of known CEUS imaging systems is challenging due to the CEUS loop length (often up to 5 minutes of imaging from contrast agent injection). As such, the burden of review in not just time but mental effort by the radiologist/clinician is reduced by the system and methods of the present teachings compared to known systems and methods.

At S226 a structured report (SR) or a free text report (FTR) are generated, and at S228, the method of reviewing the CEUS image data is complete.

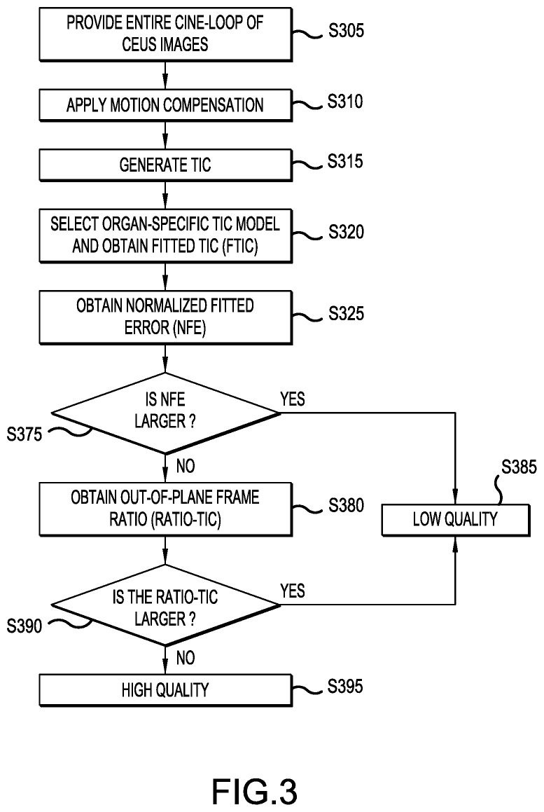

FIG. 3 illustrates a method for contrast-enhanced ultrasound image quality control, in accordance with a representative embodiment.

The method of FIG. 3 starts at S305 when an entire cine-loop (or a multiple cineloops spanning the time range of interest) of CEUS images is provided.

A targeted suspected lesion may be identified from the cine-loop either manually or automatically. The suspected lesion may be determined from a pre-contrast B-mode image with high Mechanical Index (MI) (for example: MI=1.3). Alternatively, the suspected lesion may be determined from a given frame of a CEUS loop, based on either the side-by-side low-MI B-mode image and/or the CEUS image. The steps of FIG. 3 after the identification of a targeted suspected lesion may be semi-automated once user input for the initial location of the targeted lesion are identified either in a regular B-mode or from CEUS mode. However, fully automatic quality check may be possible if the targeted lesion is automatically detected, by using techniques such as deep learning.

At S310, motion compensation is applied. Motion compensation may be applied in a relative larger region covering the targeted suspected lesion, or the whole image if necessary.

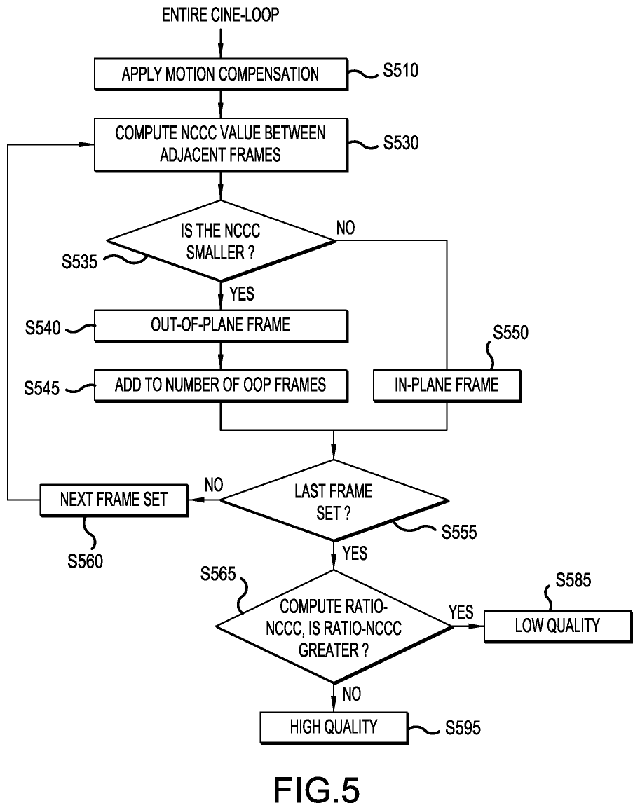

A sub-total number of out-of-plane frames may then be determined. The out-of-plane frames may be identified by a normalized cross correlation coefficient (NCCC) method between adjacent frames as described more fully below in connection with FIG. 5. The out-of-plane frames may be determined based on a comparison with a threshold for normalized cross correlation coefficients.

At S315, a TIC is generated. The raw TIC curve may be generated for a relatively small region of interest (ROI) that encompasses the suspected lesion based on the entire motion compensated CEUS loop.

At S320, an organ-specific TIC model is selected and a fitted TIC (FTIC) is obtained. While the teachings herein are particularly applicable to hepatocellular carcinoma (HCC) diagnosis, the teachings herein are extendable to other clinical applications insofar as the use of organ-specific standardized TIC models may be made available to clinicians. After generating the raw TIC curve at S315, a suitable organ-specific “standardized” model is applied to the original TIC (OTIC) curve as the fitted TIC (FTIC). Table 1 below illustrates five organ-specific models which are best fit to five different organs.

Table 1 above defines perfusion models and shows the context in which different perfusion models are applicable. The area under the ROC curve (AUC) is used to generate a distribution function in each perfusion model. The perfusion parameter of the mean transit time (MTT) is determined on a different basis for each perfusion model.

At S325, a normalized fitted error (NFE) is obtained. The normalized fitting error (NFE) may be computed as sum[abs(OTIC−FTIC)]/(N*max(FTIC)) where N is the total number of frames within the loop under to be analyzed. Notably, other normalization techniques are contemplated, including but not limited to normalizing by the sum of all FTIC's or OTIC's. Just by way of illustration such sums include, but are not limited to: sum[abs(OTIC−FTIC)]/sum (FTIC) or sum[abs(OTIC−FTIC)]/sum (OTIC).

At S375, a determination is made as to whether the NFE obtained at S325 is larger than a first threshold. If the NFE is larger than pre-determined NEF threshold (for example: 60%), the CEUS exam is considered as low-quality.

If the NFE is not larger than the first threshold (S375=No), the out-of-plane frame ratio (ratio-TIC) may be obtained at S380. The fitted difference may be computed as: diff=abs(OTIC(n)−FTIC(n)), where n is frame number starting from 1 to N (the last one); std is the standard deviation of the diff. If the OTIC curve value(n) at every temporal point is outside the pre-determined range, then this frame is considered as out-of-plane frame. The pre-determined range may be for example: [FTIC value(n)−2*std to FTIC value(n)+2*std]).

The out-of-plane frame ratio (ratio-TIC) is computed as the ratio between sub-total of out-of-plane frames to the total number of frames at the loops.

If the NFE is larger than the first threshold (S375=Yes), the CEUS images are classified as low quality at S385.

After S380, another determination is made as to whether the ratio-TIC is larger than a second threshold at S390. If the ratio-TIC is larger than the second threshold (S390=Yes), the CEUS images are classified as low quality at S385. For example, if the ratio-TIC is over a pre-determined ratio-TIC threshold such as 40%, then the loop is considered as low quality.

If the ratio-TIC is not larger than the second threshold (S390=No), the CEUS images are classified as high quality at S395. A score for the ratio-TIC may be displayed on the display 180 as a result of the processing in FIG. 3. A score may be shown between 0 and 100, for example. The score may be calculated based on the distance from the second threshold. For example, the further a ratio-TIC is below the second threshold, the closer the score is to 100, and the further a ratio-TIC is above the second threshold, the closer the score is to 0.

If the TIC analysis is not particularly suitable, such as for patients with lesions with rim enhancement), the NCCC method described herein may be used without using the TIC method. Alternatively, the out-of-plane frame ratio-TIC and the out-of-plane frame ratio-NCCC may be combined to obtain an integrated parameter through logistic regression analysis to identify specific border cases for either the TIC method or the NCCC method. When the TIC method and the NCCC method are combined, a pre-determined cut-off value may be obtained for the integrated parameter through large sample analysis.

Although not shown in FIG. 3, a TIC curve may be provided to an end user along with a high-quality cine-loop through a communication system such as a hospital PACS/HIS.

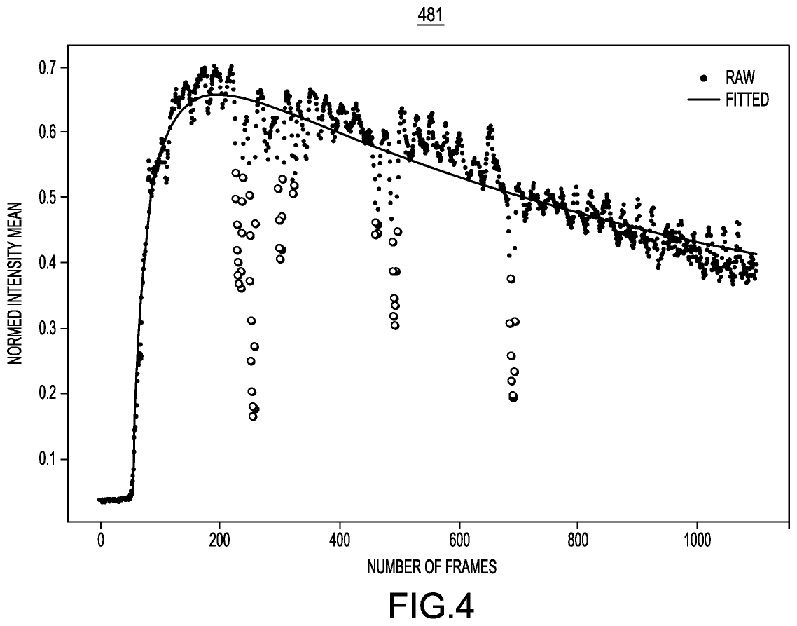

FIG. 4 illustrates a visualization of an example of a number of detected out-of-frame planes relative to a number of frames in contrast-enhanced ultrasound image quality control, in accordance with a representative embodiment.

The visualization in FIG. 4 is an example classified as high quality. In FIG. 4, the circles that drop below the main grouping of dots after 200 frames, at about 500 frames and at about 700 frames are those detected as out-of-plane frames from a large fitted error over 2 standard deviations.

FIG. 5 illustrates another method for contrast-enhanced ultrasound image quality control, in accordance with a representative embodiment.

The method of FIG. 5 involves computing the out-of-plane ratio (ratio-NCCC) as the ratio between sub-total out-of-plane frames to the total number of frames within the entire cine-loop. The out-of-plane ratio (ratio-NCCC) is computed between the number of out-of-plane frames and a total number of frames in the motion compensated loop of the CEUS images.

The method of FIG. 5 starts at S510 by applying motion compensation to an entire cine-loop.

At S530, an NCCC value between adjacent frames is computed. The NCCC value of two adjacent frames may be computed for the targeted lesion region based on the entire motion compensated CEUS loop.

At S535, a determination is made as to whether the NCCC value is smaller than a third threshold. The out-of-plane frames may be determined based on a comparison using a pre-determined NCCC threshold such as 0.7.

If the NCCC value is smaller than the third threshold (S535=No), one or both frames are classified as in-plane at S550. If the value of NCCC is below the NCCC threshold, the frame is classified as out-of-plane at S540, and the one or both frames are added to the number of out-of-plane (OOP) frames at S545.

After the classification at S545, a determination is made at S555 whether the adjacent frame(s) which were just classified are the last frame set.

If the recently-classified frames are not the last frame set (S555=No), at S560 the next frame set is selected as inputs to S530, and the process from S530 repeats.

If the recently-classified frames are the last frame set (S555=Yes), at S565 a ratio-NCCC is computed as the ratio of OOP frames divided by the total number of frames, for example. A determination is then made whether the ratio-NCCC is greater than a fourth predetermined ratio. The determination at S565 may involve determining whether the frames are low quality based on having a high out-of-plane ratio-NCCC. For example, a pre-determined ratio-NCCC threshold of 40% may be used in the determination at S555. If the ratio-NCCC is greater than the fourth predetermined ratio (S565=Yes), the CEUS images are classified as low quality at S585. Otherwise (S565=No), the CEUS images are classified as high quality at S595 if the ratio-NCCC is not greater than the fourth predetermined ratio. In some embodiments, before the CEUS images can be classified as high quality at S595, the process may include performing some or all of the checks from FIG. 3 if not already performed.

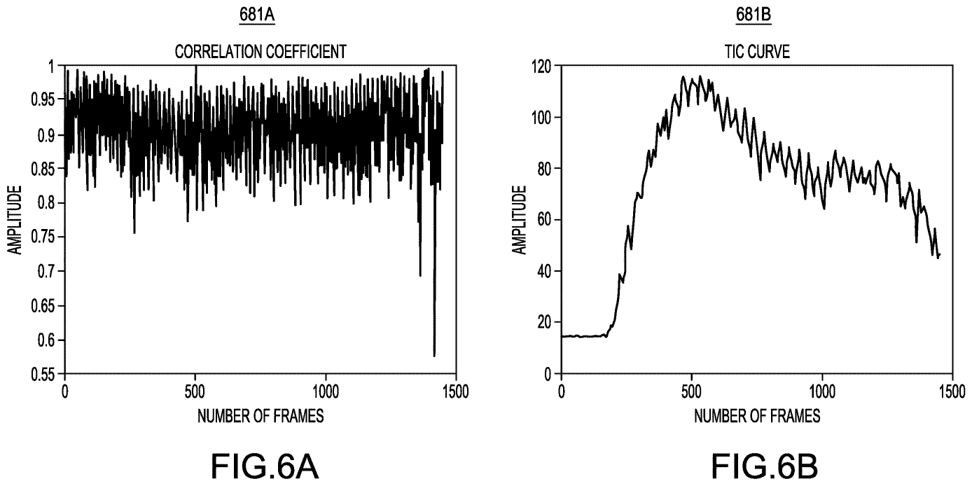

FIG. 6A illustrates a visualization of correlation coefficient relative to a number of frames in contrast-enhanced ultrasound image quality control, in accordance with a representative embodiment. FIG. 6B illustrates a visualization of a time-intensity curve relative to a number of frames in contrast-enhanced ultrasound image quality control, in accordance with a representative embodiment.

FIG. 6A and FIG. 6B illustrate visualizations for a high-quality case in which only four frames with NCCC values are below 0.7, so the out-of-plane ratio-NCCC is less than 1%. The Y axis in FIG. 6B, which plots the signal intensity vs the number of frames, exhibits relatively small fluctuations as a function of time due to the relatively high number of correlation coefficients in FIG. 6A. The example of FIG. 6A and FIG. 6B corresponds to a high quality at S595 based on the determination from S565.

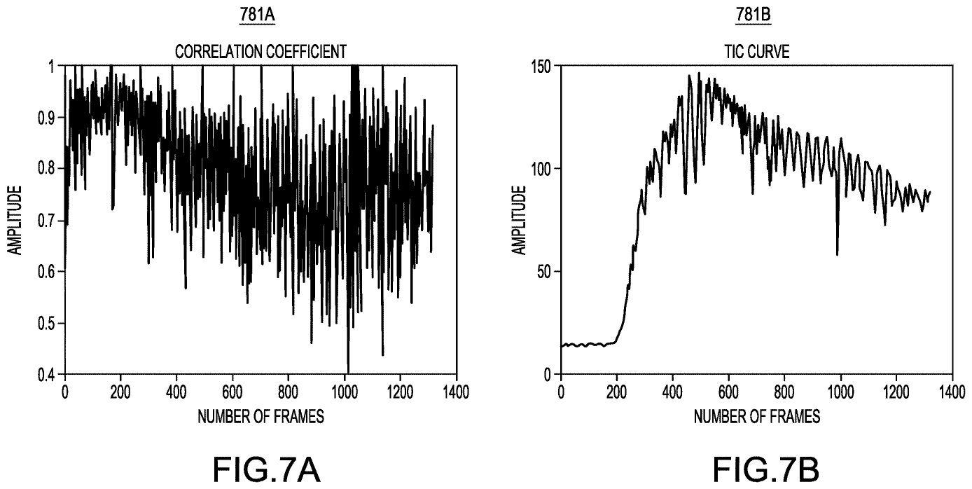

FIG. 7A illustrates another visualization of correlation coefficient relative to a number of frames in contrast-enhanced ultrasound image quality control, in accordance with a representative embodiment. FIG. 7B illustrates another visualization of a time-intensity curve relative to a number of frames in contrast-enhanced ultrasound image quality control, in accordance with a representative embodiment.

FIG. 7A and FIG. 7B illustrate visualizations for a low-quality case in which five hundred frames have NCCC values below 0.7, so the out-of-plane ratio-NCCC is over 40%. The Y axis in FIG. 7B, which plots the signal intensity vs the number of frames, exhibits relatively larger fluctuations as a function of time due to the relatively small number of correlation coefficients in FIG. 7A. The example of FIG. 7A and FIG. 7B corresponds to a low quality at S585 based on the determination from S565.

FIG. 8 illustrates a computer system, on which a method for contrast-enhanced ultrasound image quality control is implemented, in accordance with another representative embodiment.

Referring to FIG. 8, the computer system 800 includes a set of software instructions that can be executed to cause the computer system 800 to perform any of the methods or computer-based functions disclosed herein. The computer system 800 may operate as a standalone device or may be connected, for example, using a network 801, to other computer systems or peripheral devices. In embodiments, a computer system 800 performs logical processing based on digital signals received via an analog-to-digital converter.

In a networked deployment, the computer system 800 operates in the capacity of a server or as a client user computer in a server-client user network environment, or as a peer computer system in a peer-to-peer (or distributed) network environment. The computer system 800 can also be implemented as or incorporated into various devices, such as a workstation that includes a controller, a stationary computer, a mobile computer, a personal computer (PC), a laptop computer, a tablet computer, or any other machine capable of executing a set of software instructions (sequential or otherwise) that specify actions to be taken by that machine. The computer system 800 can be incorporated as or in a device that in turn is in an integrated system that includes additional devices. In an embodiment, the computer system 800 can be implemented using electronic devices that provide voice, video or data communication. Further, while the computer system 800 is illustrated in the singular, the term “system” shall also be taken to include any collection of systems or sub-systems that individually or jointly execute a set, or multiple sets, of software instructions to perform one or more computer functions.

As illustrated in FIG. 8, the computer system 800 includes a processor 810. The processor 810 may be considered a representative example of a processor of a controller and executes instructions to implement some or all aspects of methods and processes described herein. The processor 810 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 810 is an article of manufacture and/or a machine component. The processor 810 is configured to execute software instructions to perform functions as described in the various embodiments herein. The processor 810 may be a general-purpose processor or may be part of an application specific integrated circuit (ASIC). The processor 810 may also be a microprocessor, a microcomputer, a processor chip, a controller, a microcontroller, a digital signal processor (DSP), a state machine, or a programmable logic device. The processor 810 may also be a logical circuit, including a programmable gate array (PGA), such as a field programmable gate array (FPGA), or another type of circuit that includes discrete gate and/or transistor logic. The processor 810 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 800 further includes a main memory 820 and a static memory 830, where memories in the computer system 800 communicate with each other and the processor 810 via a bus 808. Either or both of the main memory 820 and the static memory 830 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 820 and the static memory 830 are articles of manufacture and/or machine components. The main memory 820 and the static memory 830 are computer-readable mediums from which data and executable software instructions can be read by a computer (e.g., the processor 810). Each of the main memory 820 and the static memory 830 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 800 further includes a video display unit 850, such as a liquid crystal display (LCD), an organic light emitting diode (OLED), a flat panel display, a solid-state display, or a cathode ray tube (CRT), for example. Additionally, the computer system 800 includes an input device 860, such as a keyboard/virtual keyboard or touch-sensitive input screen or speech input with speech recognition, and a cursor control device 870, such as a mouse or touch-sensitive input screen or pad. The computer system 800 also optionally includes a disk drive unit 880, a signal generation device 890, such as a speaker or remote control, and/or a network interface device 840.

In an embodiment, as depicted in FIG. 8, the disk drive unit 880 includes a computer-readable medium 882 in which one or more sets of software instructions 884 (software) are embedded. The sets of software instructions 884 are read from the computer-readable medium 882 to be executed by the processor 810. Further, the software instructions 884, when executed by the processor 810, perform one or more steps of the methods and processes as described herein. In an embodiment, the software instructions 884 reside all or in part within the main memory 820, the static memory 830 and/or the processor 810 during execution by the computer system 800. Further, the computer-readable medium 882 may include software instructions 884 or receive and execute software instructions 884 responsive to a propagated signal, so that a device connected to a network 801 communicates voice, video or data over the network 801. The software instructions 884 may be transmitted or received over the network 801 via the network interface device 840.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, contrast-enhanced ultrasound image quality control enables improvement of clinical practices via a quality check module for contrast-enhanced ultrasound workflow, and this is particularly applicable for the diagnosis of liver lesions. The quality check module may reduce the workload for ultrasound doctors/sonographers during CEUS examination and for radiologists during CEUS examination review and report. The time and effort for a CEUS examination procedure for both operator (ultrasound doctor or sonographer) and patient may be reduced insofar as the quality check module helps guarantee the quality of each CEUS examination, as it can help ultrasound doctors/sonographers keep the ultrasound probe still and stable while ensuring the patient is controlling his/her breathing well during the entire examination; helps the ultrasound doctors/sonographers determine when another contrast agent injection is required before the patient leaving the exam room, which will potentially save a patient recall exam; and help save the time of radiologists as only high quality CEUS loops are transferred to the picture archiving and communication system (PACS).

The teachings herein may be applied to any soft-tissue organs where tumor characterization is required. While HCC diagnosis is one of the primary clinical applications of these teachings, the quality check module for CEUS described herein may be applied to other liver lesions. Additionally, the quality check module may be extended to other organs such as thyroid or the breast by selecting a suitable fitting model for the organ of interest.

Although contrast-enhanced ultrasound image quality control has been described with reference to several exemplary embodiments, it is understood that the words that have been used are words of description and illustration, rather than words of limitation. Changes may be made within the purview of the appended claims, as presently stated and as amended, without departing from the scope and spirit of contrast-enhanced ultrasound image quality control in its aspects. Although contrast-enhanced ultrasound image quality control has been described with reference to particular means, materials and embodiments, contrast-enhanced ultrasound image quality control is not intended to be limited to the particulars disclosed; rather contrast-enhanced ultrasound image quality control extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A system for classifying ultrasound images, comprising:
a processor;
a tangible, non-transitory computer-readable medium that stores instructions, which when executed by the processor cause the system to:
apply motion compensation to a region covering a targeted suspected lesion in each ultrasound frame of interest among Contrast-Enhanced Ultrasound (CEUS) images to produce motion-compensated ultrasound frames of interest to ensure that the lesion remains at approximately the same image location for all ultrasound frames of interest among the CEUS images;
generate a time-intensity curve (TIC) based on the motion-compensated ultrasound frames of interest among the CEUS images;
apply an organ-specific TIC model to the TIC to obtain a fitted TIC (FTIC);
compute a normalized fitting error (NFE) based on a difference between the TIC and the FTIC;
compare the normalized fitting error to a first predetermined threshold;
when the normalized fitting error is larger than the first predetermined threshold, classify the CEUS images as low-quality;
when the normalized fitting error is not larger than the first predetermined threshold, obtain an out-of-plane frame ratio (ratio-TIC);
compare the out-of-plane frame ratio to a second predetermined threshold;
when the out-of-plane frame ratio is larger than the second predetermined threshold, classify the CEUS images as low-quality; and
when the out-of-plane frame ratio is not larger than the second predetermined threshold, classify the CEUS images as high-quality.

2. The system of claim 1, wherein, when executed by the processor, the instructions further cause the system to:
execute a cross-correlation coefficient algorithm to determine the out-of-plane frame ratio.

3. The system of claim 1, wherein, when executed by the processor, the instructions further cause the system to:
determine a number of out-of-plane frames by obtaining a normalized cross correlation coefficient (NCCC) between adjacent frames.

4. The system of claim 3, wherein when executed by the processor, the instructions further cause the system to:
determine the normalized cross correlation coefficient between adjacent frames based on a motion compensated loop of the CEUS images;
determine the out-of-plane frames based on a comparison with a third threshold for normalized cross correlation coefficients; and
compute an out-of-plane ratio (ratio-NCCC) between the number of out-of-plane frames and a total number of frames in the motion compensated loop of the CEUS images.

5. The system of claim 4, wherein when executed by the processor, the instructions further cause the system to:
compare the out-of-plane ratio to a fourth predetermined threshold;
when the out-of-plane ratio is not larger than the fourth predetermined threshold, classify the CEUS images as low-quality.

6. A tangible, non-transitory computer-readable medium that stores instructions, which when executed by a processor, cause the processor to:
apply motion compensation to a region covering a targeted suspected lesion in each ultrasound frame of interest among Contrast-Enhanced Ultrasound (CEUS) images to produce motion-compensated ultrasound frames of interest to ensure that the lesion remains at approximately the same image location for all ultrasound frames of interest among the CEUS images;
generate a time-intensity curve (TIC) based on the motion-compensated ultrasound frames of interest among the CEUS images;
apply an organ-specific TIC model to the TIC curve to obtain a fitted TIC (FTIC);
compute a normalized fitting error (NFE) based on a difference between the TIC and the FTIC;
compare the normalized fitting error to a first predetermined threshold;
when the normalized fitting error is larger than the first predetermined threshold, classify the CEUS images as low-quality;
when the normalized fitting error is not larger than the first predetermined threshold, obtain an out-of-plane frame ratio (ratio-TIC);
compare the out-of-plane frame ratio to a second predetermined threshold;
when the out-of-plane frame ratio is larger than the second predetermined threshold, classify the CEUS images as low-quality; and
when the out-of-plane frame ratio is not larger than the second predetermined threshold, classify the CEUS images as high-quality.

7. The tangible, non-transitory computer-readable medium of claim 6, wherein the instructions, when executed by the processor further cause the processor to:
execute a cross-correlation coefficient algorithm to determine the out-of-plane frame ratio.

8. The tangible, non-transitory computer-readable medium of claim 6, wherein the instructions, when executed by the processor further cause the processor to:
determine a number of out-of-plane frames by obtaining a normalized cross correlation coefficient (NCCC) between adjacent frames.

9. The tangible, non-transitory computer-readable medium of claim 8, wherein the instructions, when executed by the processor to compare TICs, further cause the processor to:
determine the normalized cross correlation coefficient between adjacent frames based on a motion compensated loop of the CEUS images;
determine the out-of-plane frames based on a comparison with a third threshold for normalized cross correlation coefficients; and
compute an out-of-plane ratio (ratio-NCCC) between the number of out-of-plane frames and a total number of frames in the motion compensated loop of the CEUS images.

10. The tangible, non-transitory computer-readable medium of claim 9, wherein the instructions, when executed by the processor to compare TICs, further cause the processor to:
compare the out-of-plane ratio to a fourth predetermined threshold;
when the out-of-plane ratio is not larger than the fourth predetermined threshold, classify the CEUS images as low-quality.

11. A method of classifying ultrasound images, the method comprising:
applying motion compensation to a region covering a targeted suspected lesion in each ultrasound frame of interest among Contrast-Enhanced Ultrasound (CEUS) images to produce motion-compensated ultrasound frames of interest to ensure that the lesion remains at approximately the same image location for all ultrasound frames of interest among the CEUS images;
generating a time-intensity curve (TIC) based on the motion-compensated ultrasound frames of interest among the CEUS images;
applying an organ-specific TIC model to the TIC to obtain a fitted TIC (FTIC);
computing a normalized fitting error (NFE) based on a difference between the TIC and the FTIC;
comparing the normalized fitting error to a first predetermined threshold;
when the normalized fitting error is larger than the first predetermined threshold, classifying the CEUS images as low-quality;
when the normalized fitting error is not larger than the first predetermined threshold, obtaining an out-of-plane frame ratio (ratio-TIC);
comparing the out-of-plane frame ratio to a second predetermined threshold;
when the out-of-plane frame ratio is larger than the second predetermined threshold, classifying the CEUS images as low-quality; and
when the out-of-plane frame ratio is not larger than the second predetermined threshold, classifying the CEUS images as high-quality.

12. The method of claim 11, further comprising:
executing a cross-correlation coefficient algorithms to determine the out-of-plane frame ratio.

13. The method of claim 11, further comprising:
determining a number of out-of-plane frames by obtaining a normalized cross correlation coefficient (NCCC) between adjacent frames.

14. The method of claim 13, further comprising:
determining the normalized cross correlation coefficient between adjacent frames based on a motion compensated loop of the CEUS images;
determining the out-of-plane frames based on a comparison with a third threshold for normalized cross correlation coefficients; and
computing an out-of-plane ratio (ratio-NCCC) between the number of out-of-plane frames and a total number of frames in the motion compensated loop of the CEUS images.

15. The method of claim 14, further comprising:
comparing the out-of-plane ratio to a fourth predetermined threshold;
when the out-of-plane ratio is not larger than the fourth predetermined threshold, classifying the CEUS images as low-quality.
